(19) United States
(12) Patent Application Publication
(10) Pub. No.: US 2009/0028949 A1
(43) Pub. Date: Jan. 29, 2009

(54) CALCIUM PHOSPHATE-BASED ADHESIVE FORMULATION FOR BONE FILLING WITH SWELLING PROPERTIES

(76) Inventors: Alain Leonard, Caixon (FR); Stephane Goncalves, Balma (FR); Juliette Fitremann, Toulouse (FR); Ariane Bercier, Creteil (FR)

Correspondence Address: MINTZ LEVIN COHN FERRIS GLOVSKY & POPEO, ONE FINANCIAL CENTER, BOSTON, MA, 02111, US

(21) Appl. No.: 11/830,065

(22) Filed: Jul. 30, 2007

(30) Foreign Application Priority Data
Jul. 25, 2007 (FR) FR 07 05405

(51) Int. Cl. A61K 9/14; A61K 33/42; A61P 19/00

(52) U.S. Cl. 424/489; 424/602

(57) ABSTRACT

The present invention relates to a calcium phosphate-based formulation for bone filling, comprising at least one adjuvant giving adhesion properties, said at least one adjuvant can be selected from the sugar and sugar derivative group.

The bone cement described in the present invention is characterised by adhesive and swelling properties particularly advantageous, improving dispersion of the biomaterial in the cavities to be filled and facilitating osteointegration.

### RELATED APPLICATION

The present application claims the benefit of priority of French Patent Application FR 07 05406, filed on Jul. 25, 2007 and incorporated by reference, herein, in its entirety.

The present invention relates to a formulation based on biomaterials useful in the orthopaedic and dental sector with a view to the filling of bone or dental defects, and the colonisation thereof with live cells of the tissue wherein they are to be implanted.

Bone substitutes are presently gradually finding their place in bone treatment and filling. Thus, even though some orthopaedists remain somewhat reluctant to use them because of their non-natural nature, numerous products are being developed and therefore marketed. At the present time, there is still no precise definition of the term biomaterial but it can be considered that it consists of a material that meets the applicable standards and regulatory requirements and enables a reinforcement of the bone structure. This material should be biotolerated, biocompatible, and if possible bioresorbable.

The materials essentially comprising calcium phosphates, have all such properties and also display osteoconductive properties. According to their formulation, forms, quantity, the resorbability is more or less rapid. Their efficacy is now acknowledeged.

Whether for the dental, orthopaedic and/or radiological field, the different practitioners need products with different forms and viscosities. Therefore, according to the applications, surgeons from all fields need products in different forms:
cylinder (solid form)
gel or paste (more or less viscous form)
cement (malleable form having a setting time before hardening).

Numerous bone substitutes are already available on the market. They exist in various forms and applications. Indeed, calcium phosphate ceramics are available that have been used for over twenty years in granules, powder, and in other geometric forms which can thus be adapted to the defects to be filled, but to the expense of a traumatising surgery. A new route has existed for some ten years, which makes use of gels or pastes, which are injectable and therefore enable mini-invasive surgery which is less traumatic for the patient. However, the forms currently available display adherence and adaptation limitations explaining their low use during reconstructive operations.

One of the aspects that has not been studied very much to date is that of the adhesion of these pastes or gels. However, this property is fundamental in the mechanisms occurring on the interfaces between the biomaterial and the bone. It is involved in bone substitute migration reactions and is reported to enable better control of product resorption. As the product has an identical chemical composition to that of the mineral phase of the bone, the desired biomimetism is obtained more homogeneously and therefore, the osteointegration of the biomaterial can only be improved. As the product is resorbed on the surface, this contact area rapidly becomes a mineralization front and subsequently new bone.

This adhesion would also enable the product to remain in the treated area and thus prevent any migration of the product into more sensitive areas. In fact, the primary application currently seems to be the dental sector where the risks of migrations are limited. The same does not apply on the vertebrae where any migration of the product is liable to have dramatic consequences.

In a complementary manner to the adhesion properties, a swelling of pastes or gels has been observed. This property enables improved dispersion of the biomaterial in the cavities to be filled. Indeed, when the product is injected, the swelling or expansion of the biomaterial improves the contact surface area between the substitute and the bone. In addition, during the swelling of the material, the injection of an active ingredient may be considered. However, this release is liable to be immediate and therefore have a flash effect. Depending on the applications, said release may prove to be of significant interest.

The aim of the present invention is to develop a gel or a paste intended for bone filling displaying enhanced adhesion and swelling qualities. In this way, such a formulation proves to be particularly practical to handle and effective in terms of bone filling. This is performed by means of a biomaterial based on calcium phosphates and specified adjuvants added in precise quantities.

In the present application, the term “adhesion” defines the property displayed by the cement paste to adhere to a substrate, as characterised and measured by the adherence tests described below. The measurements made give access to two quantitative values for this property, the adhesion strength and energy. This property may also be referred to as “pegosity” (immediate adhesion in contact with substrate). The terms “adhesive” (in the common sense) or “adherence” (tests characterising this adhesion) may also be used to refer to the property observed.

Of the calcium phosphates used in the field of biomaterials for bone filling, hydroxyapatite is frequently used for its characteristics similar to the mineral phase of bone. Hydroxyapatite is considered to be non-resorbable when in its crystallised and therefore well calcined form. If this phase is poorly crystallised (it is in the form of an amorphous phase), it is very highly resorbable. In fact, the solubility coefficient of this poorly crystallised hydroxyapatite is markedly higher than calcined hydroxyapatite but also than all the products from tricalcium phosphate ceramics, currently very popular in the dental field during to their rapid and effective resorption.

Another calcium phosphate frequently used in the dental field is beta tricalcium phosphate. One of the advantages of these products is being totally synthetic.

Atypically, the applicant had the idea of studying a particular class of adjuvants liable to be incorporated in a formulation meeting the pre-defined aims belonging to the sugar derivative group. It particularly consists of sucrose fatty esters (sucroesters), which are in fact known compounds, currently produced on an industrial scale (approximately 5000 T/year) for their use essentially as food emulsifiers (E 473), in pharmaceuticals and in cosmetics. They are biocompatible, biodegradable, non-toxic products (acute oral toxicity >2 g/kg/day). The grafting of a sugar type head onto fatty acids reduces the excessively detergent, and therefore irritant, nature very significantly, when used in the surfactant “soap” form. Compared to other non-ionic surfactant derivatives (ethylene oxide derivatives), they display a better toxicological profile.

In terms of their structure, the polar head is formed by a sucrose (table sugar) molecule, whereon fatty acids are grafted via an ester bond. The number of grafted chains may vary from 1 to 8, usually from 1 to 5 (degree of substitution), and the chain length generally from 12 to 18 carbons (C12: laurates, C16: palmitates, C18: stearates). These compounds are marketed in the form of regioisomer mixtures, with variable degrees of substitution and more or less pure chain lengths.

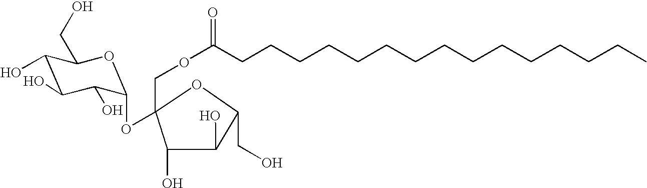

The greater the number of grafted chains and the longer the fatty chain, the more the sucrose fatty ester will be hydrophobic, slightly soluble in water. Laurates, palmitates, and possibly stearates with low degrees of substitution (80% monoesters) display a good solubility in water. The most substituted batches are progressively less and less soluble and in this case form gels followed by dispersions.

Another characteristic of these molecules is their ability to form lyotropic phases in the presence of water, i.e. organised phases. These solutions, according to their concentration, may display gel behaviours, or, in the broadest sense, viscoelastic properties of interest (C. Calahorro, J. Munoz, M. Berjano, A. Guerrero, C. Gallegos, JAOCS, 1992, 69, (7), 660-666. “Flow behavior of sucrose stearate/water systems”).

Little research has been conducted to study the incorporation of sugar derivatives in calcium phosphate-based biomaterials. Nevertheless, it is possible to mention Marc BOHNER, Patent application No. WO 2004/000374 followed by Patent application No. WO 2005/084726 and that of M. P. GINEBRA et al, Patent application No. WO 2006/030054. These applications display the lubricant and emulsifying properties of surfactants. In this way, they are cited for their injectability and those of macropore creation and therefore porosity.

A final patent application refers to the texture of the final product, i.e. a paste. It consists of the Russian application “Preparation of ostim apatite for stimulating growth in bone tissue”, inventors Nikolaevich RUDIN, Victor BOZHEVOLNOV, Vladislav ZUEV et al referring to a hydroxyapatite nanoparticle gel, free from any additive.

The present invention relates to an injectable pasty or gelled form, offering adhesion and swelling properties such that it enables particularly advantageous handling and application on the implantation site, eventually reducing procedure-related trauma, using mini-invasive surgery.

The formulation according to the invention consists of the addition of at least one sugar or sugar derivative to a calcium phosphate mixture. It was found that the addition of certain adjuvants enabled a better homogeneity of the mixture and made it possible to obtain the selected physical state. In this way, surprisingly, these mixtures thus displayed adhesion and swelling properties that had never been described in the prior art.

In fact, the formulation according to the invention displays an adhesion power along with swelling properties that can be adjusted according to the choice of adjuvants from the selected list and also the percentage of adjuvant introduced.

The properties of the formulations obtained in this way were evaluated by means of tests used to characterise the adhesion and swelling capabilities of gels and pastes. The principle of these tests is explained below:

The present invention will be understood more clearly using the following figures:

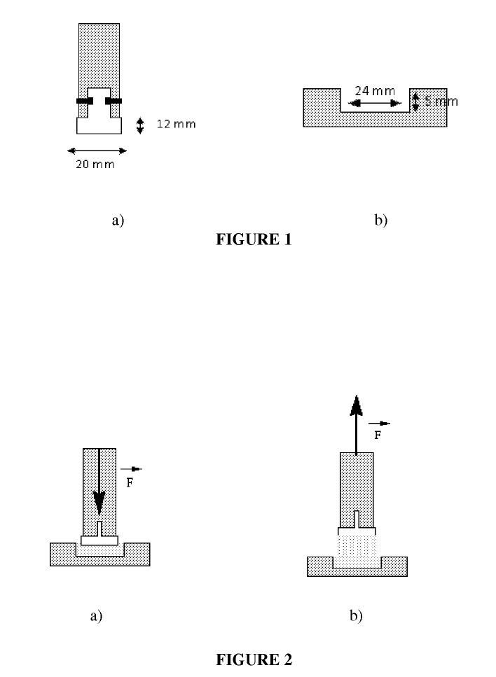

FIG. 1: mobile device used to perform adhesion tests, a) mobile head, b) trough.

FIG. 2: operation of mobile device during an adhesion test of a formulation according to the invention, a) compression of formulation, b) traction of formulation.

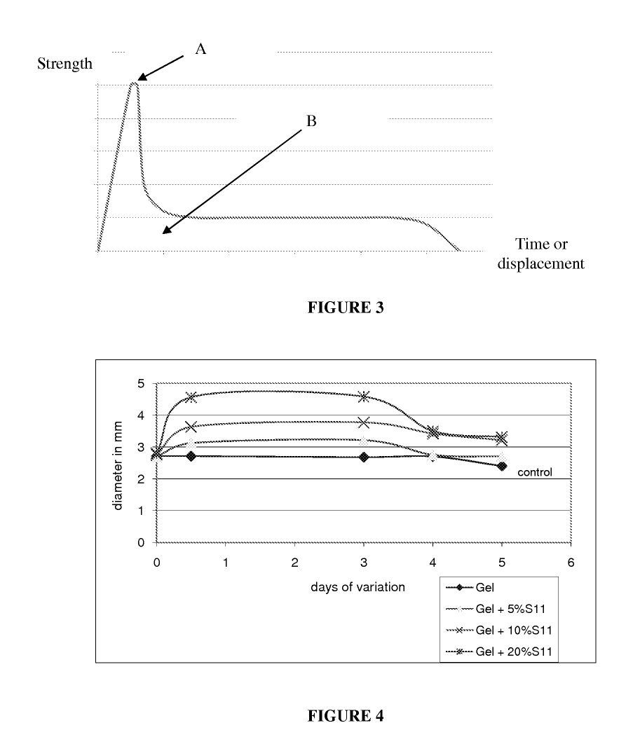

FIG. 3: standard adhesion curve with A corresponding to the Adhesion strength and B corresponding to the Adhesion Energy.

The present invention was embodied in various formulations in gel or paste form wherein the swelling and adhesion effects were characterised by the following figures:

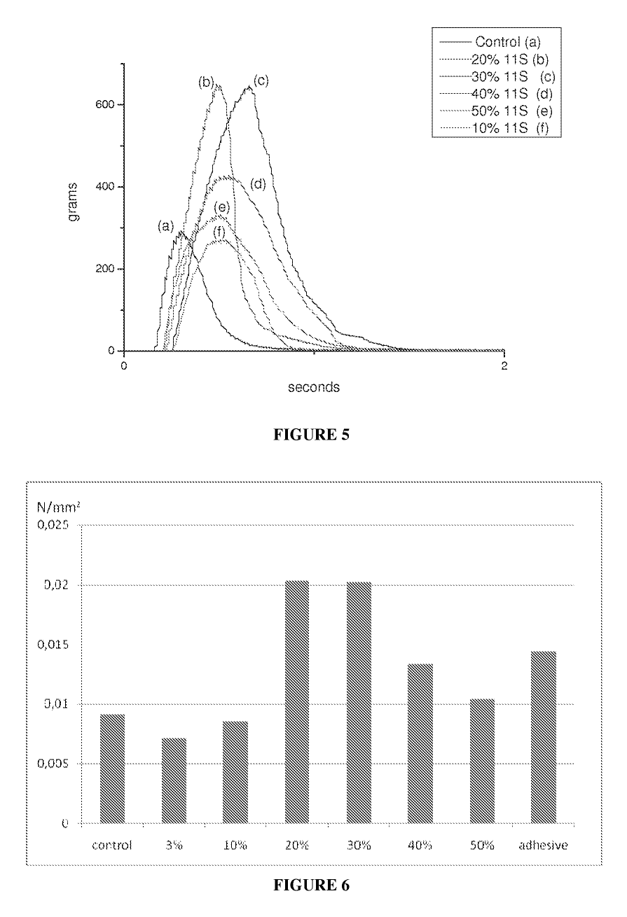

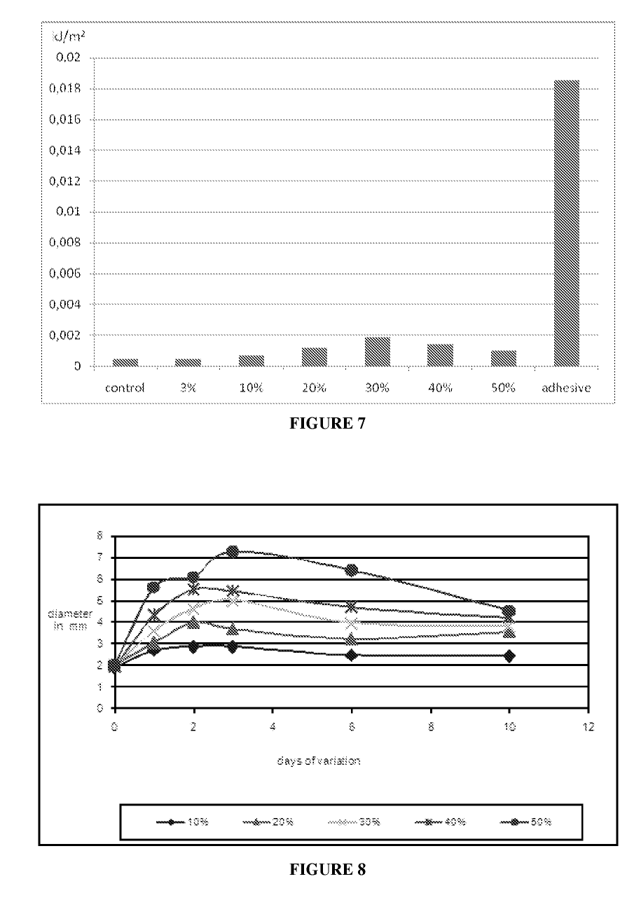

formulation 1 according to the invention in gel form: FIG. 4 corresponding to the swelling curves, FIG. 5 corresponding to the adhesion curves, FIG. 6 corresponding to the adhesion strength, FIG. 7 corresponding to the adhesion energies.

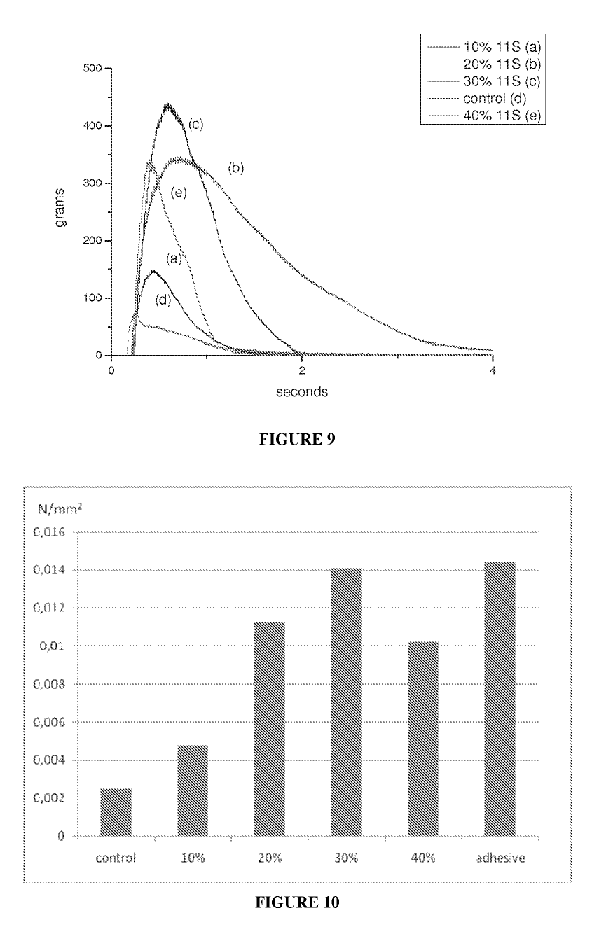

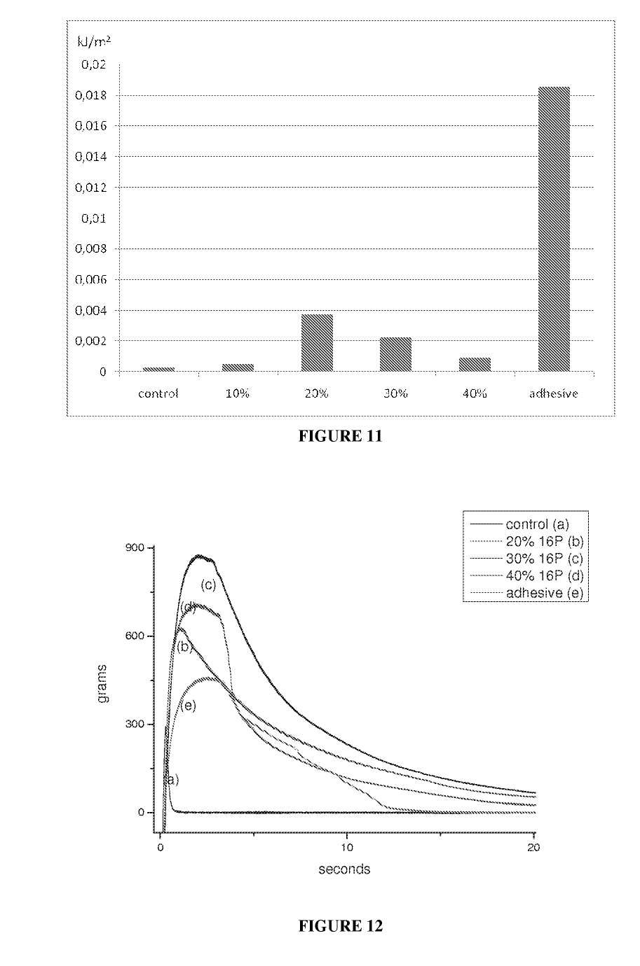

formulation 2 according to the invention in paste form: FIG. 8 corresponding to the swelling curves, FIG. 9 corresponding to the adhesion curves, FIG. 10 corresponding to the adhesion strength, FIG. 11 corresponding to the adhesion energies.

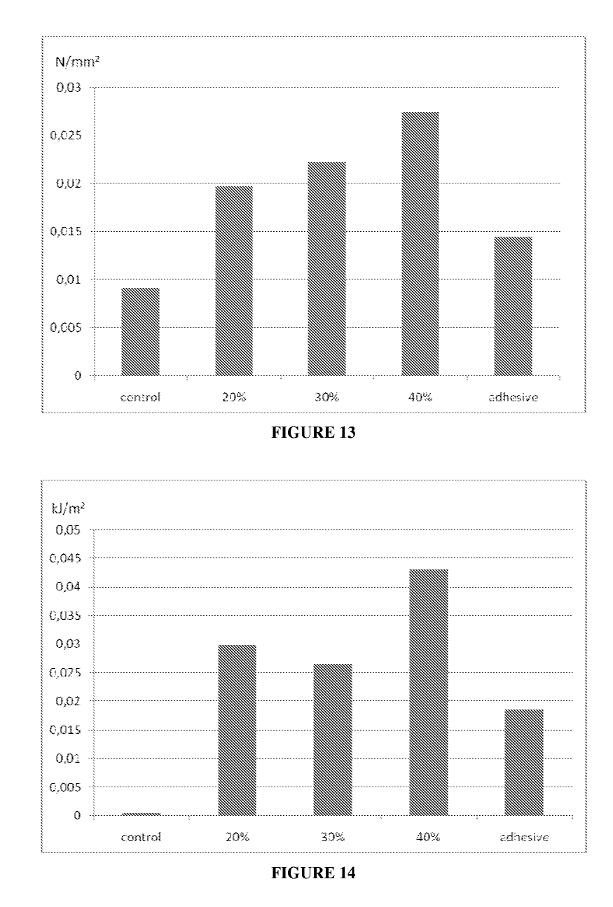

formulation 3 according to the invention in gel form: FIG. 12 corresponding to the adhesion curves, FIG. 13 corresponding to the adhesion strength, FIG. 14 corresponding to the adhesion energies.

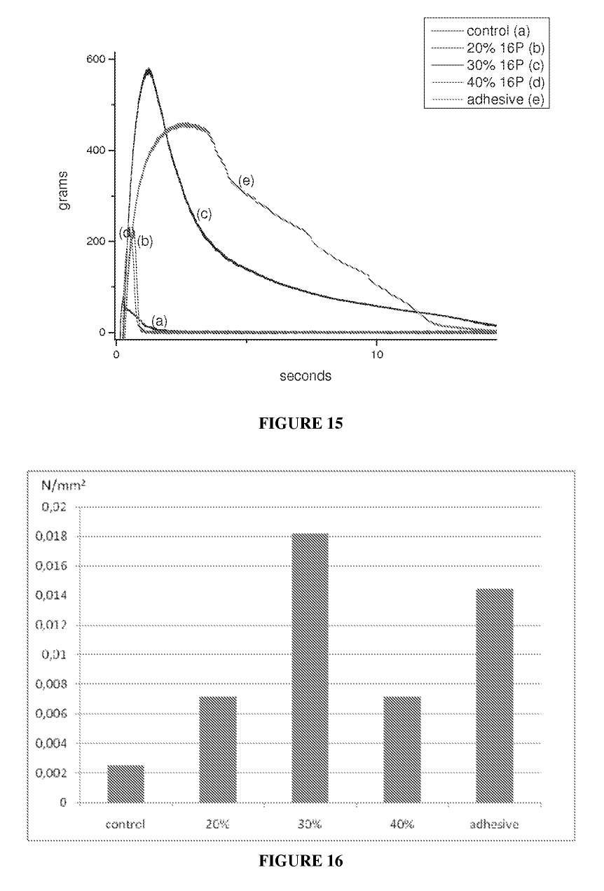

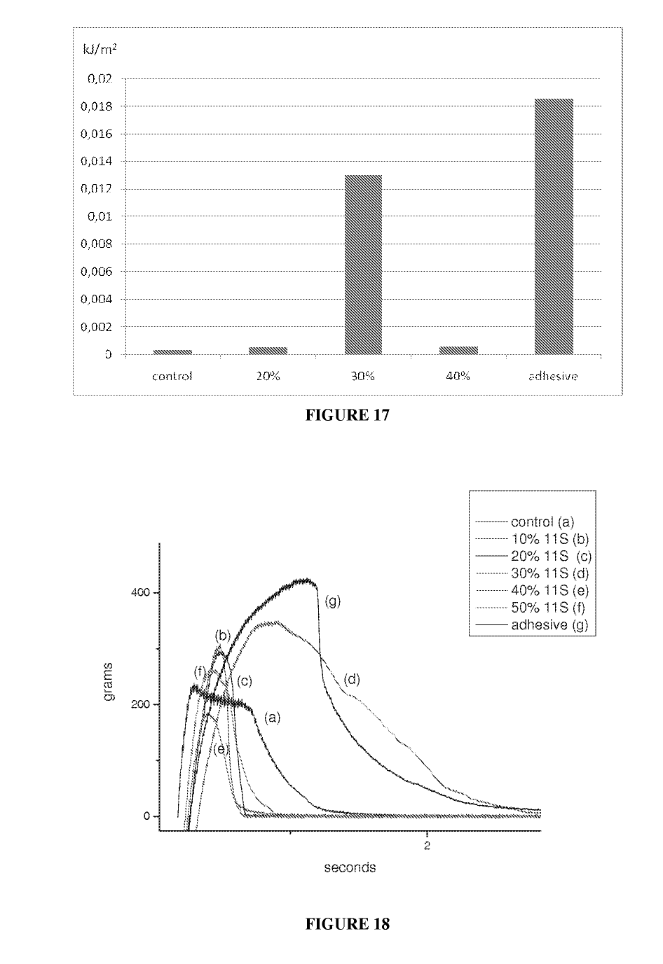

formulation 4 according to the invention in paste form: FIG. 15 corresponding to the adhesion curves, FIG. 16 corresponding to the adhesion strength, FIG. 17 corresponding to the adhesion energies.

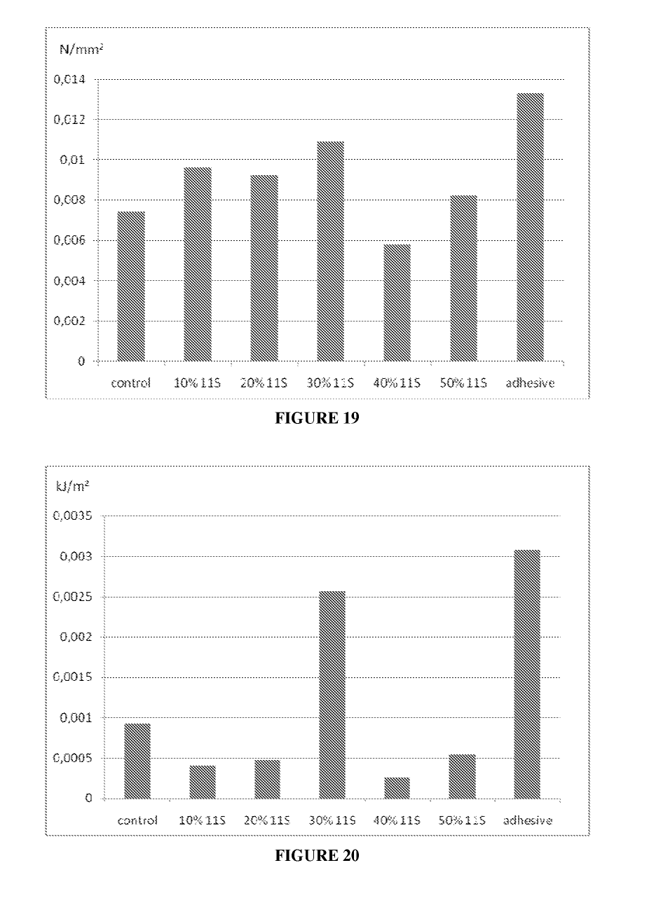

formulation 5 according to the invention in gel form: FIG. 18 corresponding to the adhesion curves, FIG. 19 corresponding to the adhesion strength, FIG. 20 corresponding to the adhesion energies.

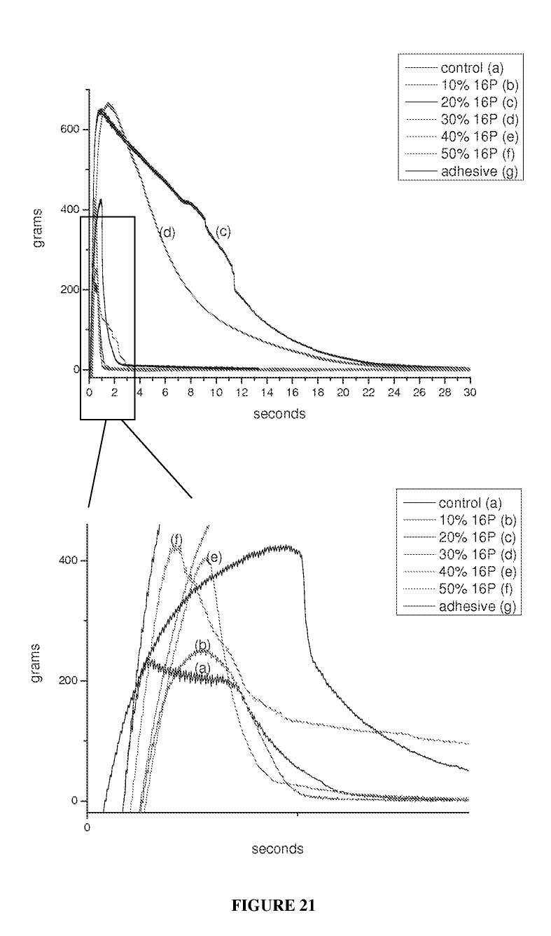

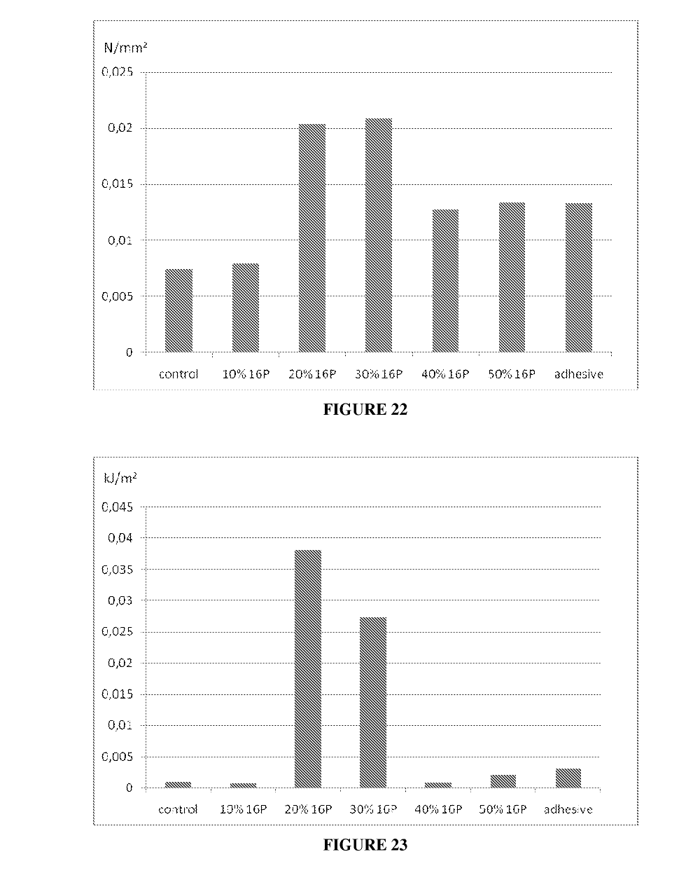

formulation 6 according to the invention in gel form: FIG. 21 corresponding to the adhesion curves, FIG. 22 corresponding to the adhesion strength, FIG. 23 corresponding to the adhesion energies.

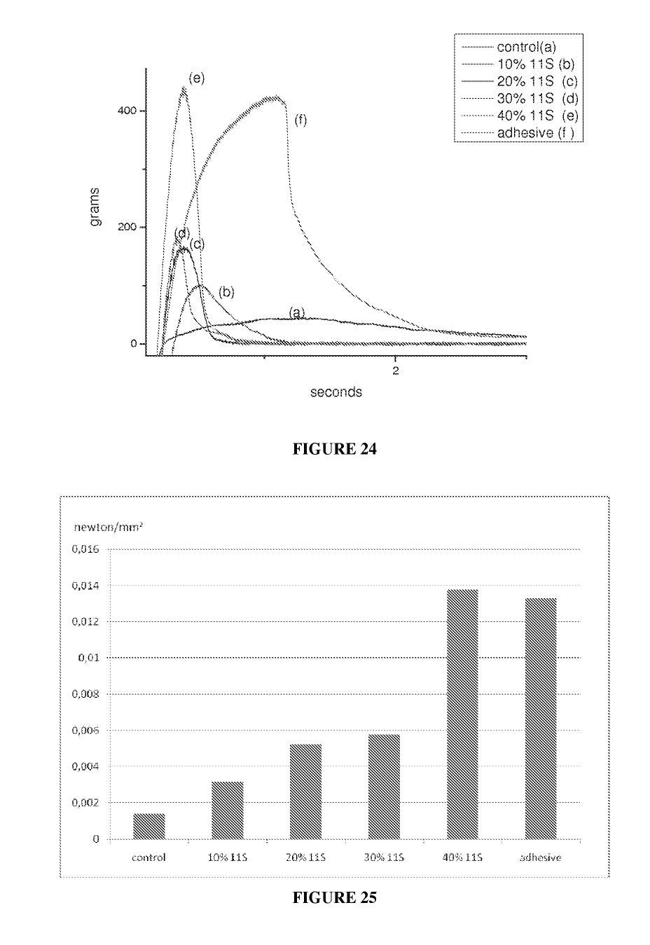

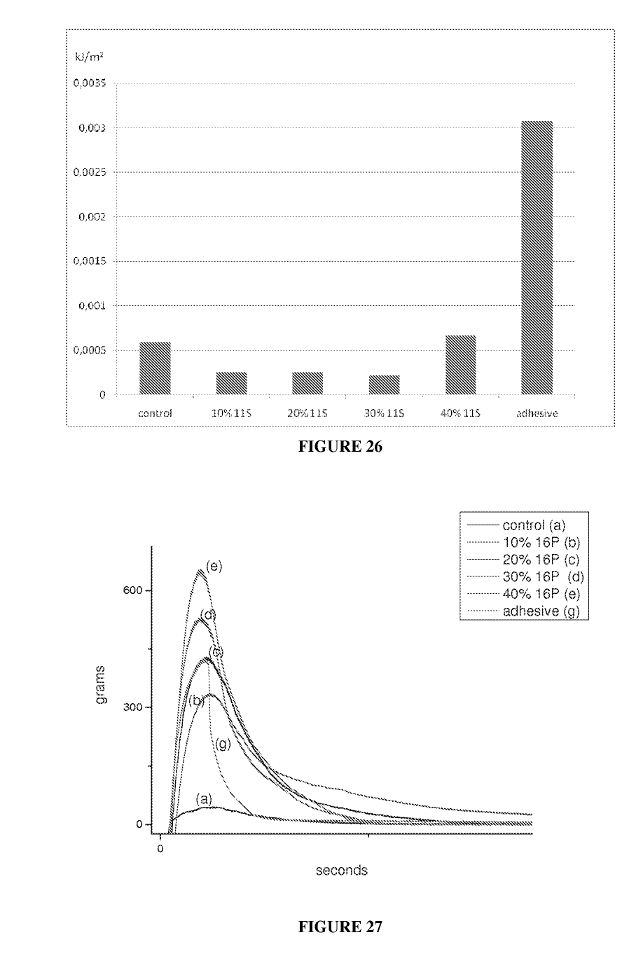

formulation 7 according to the invention in paste form: FIG. 24 corresponding to the adhesion curves, FIG. 25 corresponding to the adhesion strength, FIG. 26 corresponding to the adhesion energies.

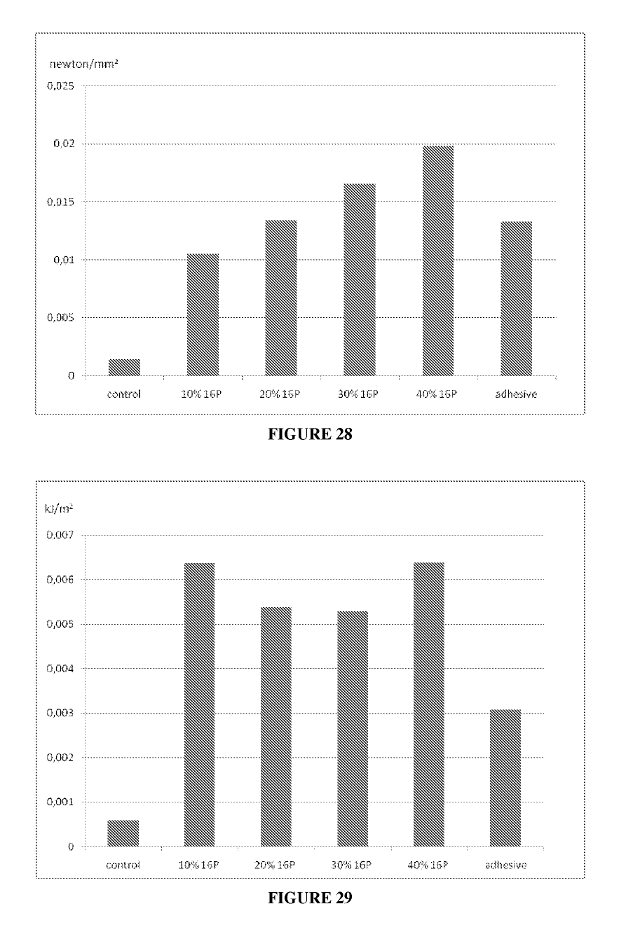

formulation 8 according to the invention in paste form: FIG. 27 corresponding to the adhesion curves, FIG. 28 corresponding to the adhesion strength, FIG. 29 corresponding to the adhesion energies.

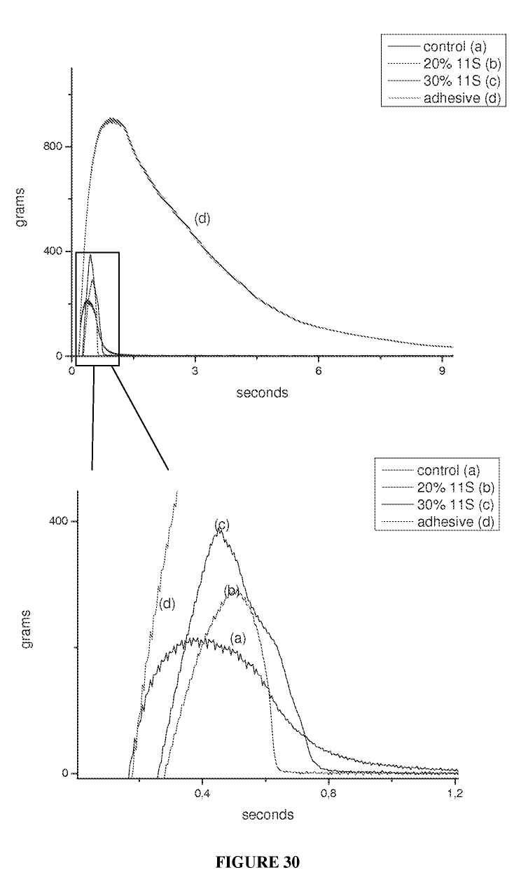

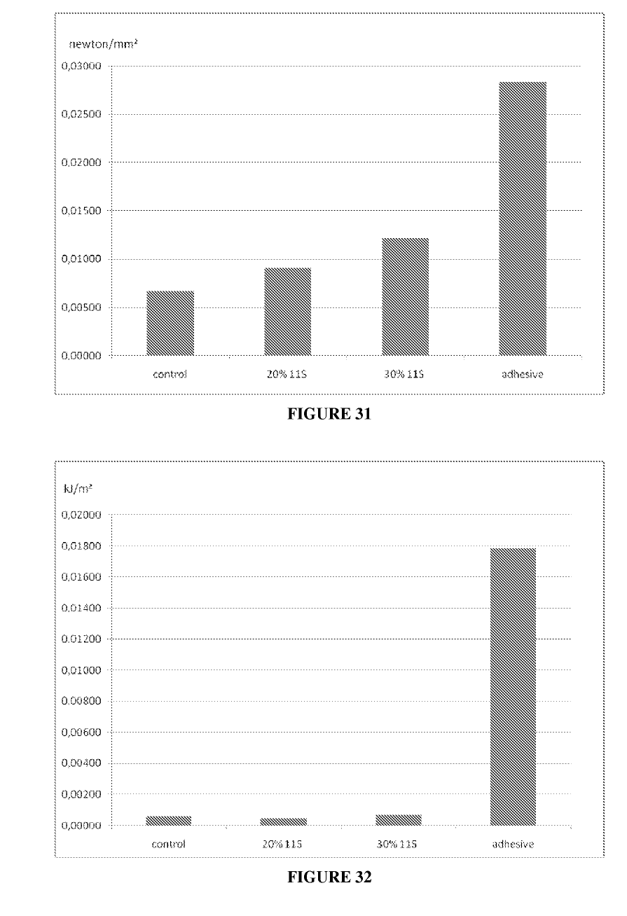

formulation 9 according to the invention in gel form: FIG. 30 corresponding to the adhesion curves, FIG. 31 corresponding to the adhesion strength, FIG. 32 corresponding to the adhesion energies.

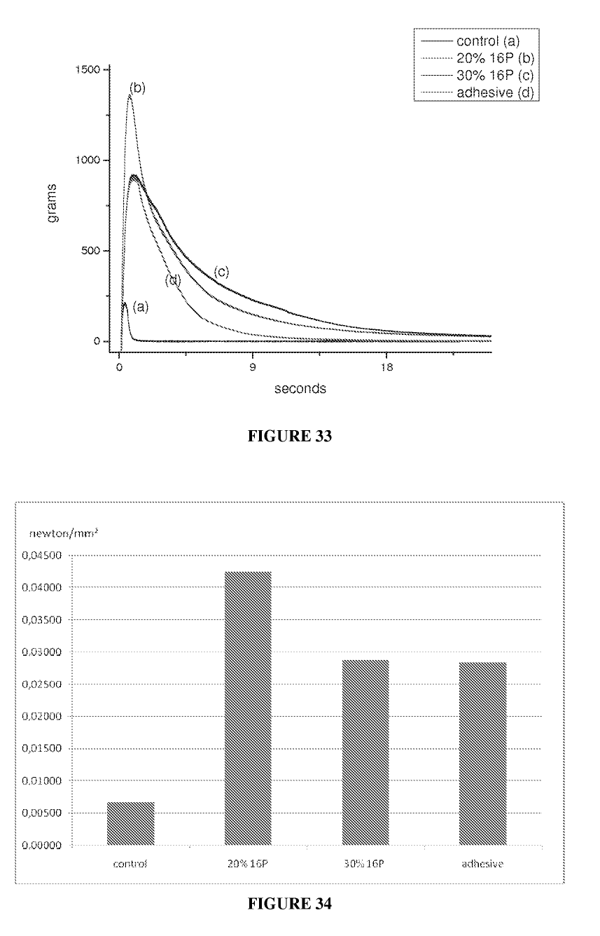

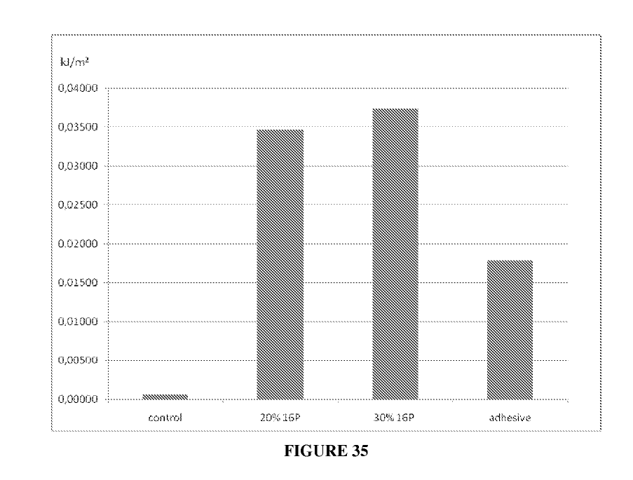

formulation 10 according to the invention in gel form: FIG. 33 corresponding to the adhesion curves, FIG. 34 corresponding to the adhesion strength, FIG. 35 corresponding to the adhesion energies.

The adhesion test performed on our formulations is of the “probe tack” type. This test was performed by means of a texturometer. The mobile device used for these tests consists of two parts: (FIG. 1)

An aluminum panel at the centre of which a flat-bottomed trough, 24 mm in diameter and 5 mm high, is machined.

An aluminum piston whereon machined 20 mm diameter “heads” of various materials can be fitted.

The various materials envisaged are: aluminum, steel, stainless steel, Plexiglas, nylon, Teflon and bone (bovine tibia).

The adhesion test follows two steps: (FIG. 2)

Firstly, the cement is mixed, introduced into the “trough” and levelled flush with the top edge. At t=5 minutes thirty, the arm of the mobile device is lowered to the level of the formulation and a force of 800 g is applied until the head of the mobile device is inserted by 2 mm into the paste, and the “head” is then kept is contact with the formulation for 20 seconds.

The second part of the experiment consists of a traction test. The arm of the mobile device is raised at a constant speed (0.2 mm/second) until it returns to the initial point.

The resistance offered by the cement varies during the test, according to its adhesive properties. Therefore, typical curves comparable to that displayed in FIG. 3 are obtained.

On the basis of these curves, two characteristic values are determined:

the adhesion strength (N/mm²) corresponding to the peak of the curve

the adhesion energy (kJ/m²) which may be related to the area under the curve.

The tests were performed comparatively between a gel and/or a paste, wherein the composition does not contain any additive listed above (Control), a gel and/or a paste containing various percentages of these additives and a commercially available white adhesive stick.

The comparative tests were performed for the different adjuvants in different percentages in the gel and/or paste, for their adhesion with respect to the different materials (aluminum, steel, stainless steel, Plexiglas, nylon, Teflon and bone (bovine tibia)).

The tests demonstrated that the forces obtained increased significantly according to the percentage of adjuvant introduced. The increase ratios are strongly linked with the percentages introduced up to a plateau. In fact, up to approximately 30% of adjuvant introduced, the adhesive effect increases significantly and then regresses if said percentage continues to increase. The values obtained are similar or even greater than those obtained with so-called “standard adhesive” sticks already on the market.

The swelling power of the formulations was also characterised by means of a test. The paste and/or gel produced is introduced into a syringe and then ejected directly into water. After the injection, the diameter of the cylinder obtained is measured and the variation of this diameter over time is then monitored.

The tests were performed comparatively between a gel and/or a paste, wherein the composition does not contain any additive listed above (Control), a gel and/or a paste containing various percentages of these adjuvants.

The introduction of adjuvant into the paste and/or gel formulation reveals swelling properties in contact with water. The surface area increase ratio is of the order of two-fold to four-fold according to the percentage introduced. In this case, the swelling is entirely linked with the percentage of adjuvants introduced. However, for percentages greater than 50%, the product no longer has any cohesion and is dispersed entirely in a wet medium. The swelling of the paste and/or gel reaches a plateau at a certain time and then declines over time.

The present invention relates to a calcium phosphate-based formulation for bone filling characterised in that it comprises at least one adjuvant giving adhesion and swelling properties.

The formulation for bone filling according to the invention particularly comprises:

at least one calcium phosphate

at least one adjuvant

with or without the addition of deionised water

The calcium phosphates used in the present invention has a Ca/P ratio between 0.4 and 2; preferentially between 1.4 and 1.8.

The selected calcium phosphates include beta tricalcium phosphate, hydroxyapatite, brushite, monetite, alpha tricalcium phosphate, tetracalcium phosphate, octocalcium phosphate. Preferentially, the formulation for bone filling will comprise beta tricalcium phosphate and/or hydroxyapatite.

The calcium phosphates will be used either directly in gel form (obtained during chemical synthesis), or in dry powder form (with a particle size distribution between 1 μm and 500 μm which, mixed with water, makes it possible to obtain a paste).

The total percentage of calcium phosphate used in the formulation according to the invention is between 15 and 99.99%.

The formulation according to the invention comprises at least one adjuvant selected from the sugar and sugar derivative group.

The selected adjuvants include sugars and sugar derivatives, more specifically monosaccharides (e.g. glucose, fructose, galactose, xylose, arabinose), disaccharides (e.g. sucrose, lactose), oligosaccharides (e.g. inulin, fructooligosaccharides, glucooligosaccharides), polysaccharides (cellulose and cellulose derivatives, hemicelluloses, starch and starch derivatives, alginates, pectins, chitosan and chitosan derivatives, dextrans and derivatives thereof) and derivatives thereof.

The selected adjuvants are preferentially sugar-derived surfactants: sorbitan esters, sucrose fatty esters (sucrose laurate, sucrose myristate, sucrose palmitate, sucrose stearate, sucrose oleate, sucrose behenate, sucrose erucate, pure or in mixtures of mono, di, tri, tetrasubstitutes and more), sucroglycerides, alkylpolyglucosides (with glucose polar head, and with octyl, decyl, dodecyl, tetradecyl, hexadecyl, octadecyl alkyl chain), alkylpolyglycosides (with polar head consisting of any type of saccharide, and with octyl, decyl, dodecyl, tetradecyl, hexadecyl, octadecyl alkyl chain).

More specifically, a sugar-derived surfactant used to obtain the formulation according to the invention is represented by a sucrose fatty ester. The percentage of this sucrose fatty ester is between 0.01 and 55%, preferentially between 10 and 40%.

The present invention is characterised in that it makes it possible to obtain a calcium phosphate-based formulation for bone filling displaying an adhesion energy up to 60 times greater than a conventional calcium phosphate-based formulation comprising no adjuvant.

It also makes it possible to obtain a formulation for bone filling displaying a swelling power up to 4 times greater than a conventional calcium phosphate-based formulation comprising no adjuvant.

### EXAMPLES

The following examples will make it possible to understand the invention more clearly, without limiting the scope thereof, however.

Formulation 1

A mixture of 3 grams of hydroxyapatite gel and the suitable amount of sucrose stearate to display a hydrophile/lipophile balance equal to 11-HLB 11-(corresponding to the ratio of the polar part of the surfactant to the apolar part), hereafter referred to as sucroester S11 (corresponding to the defined percentage) was prepared. The mixture was then introduced in the syringe to be injected into water in order to measure the diameter.

The results below show the variation of the swelling of the gel in the event of the introduction of sucroester S11 up to a percentage of 20% into the hydroxyapatite gel.

| | Days of variation | | | | | | | | | |
|---|---|---|---|---|---|---|---|---|---|---|
| | 0 | | 0.5 | | 3 | | 4 | | 5 | |
| % S11 | Ø | S | Ø | S | Ø | S | Ø | S | Ø | S |
| 0 | 2.71 | 5.77 | 2.71 | 5.77 | 2.68 | 5.64 | 2.7 | 5.73 | 2.4 | 4.52 |
| 5 | 2.68 | 5.64 | 3.12 | 7.65 | 3.21 | 8.09 | 2.75 | 5.94 | 2.7 | 5.73 |
| 10 | 2.75 | 5.94 | 3.63 | 10.35 | 3.77 | 11.16 | 3.42 | 9.19 | 3.21 | 8.09 |
| 20 | 2.8 | 6.16 | 4.56 | 16.33 | 4.58 | 16.47 | 3.5 | 9.62 | 3.31 | 8.60 |

Ø = diameter of section (in mm)
S = surface area of section (en mm²)

A mixture of 3 grams of hydroxyapatite gel and the suitable amount of sucroester S11 (corresponding to the defined percentage) was prepared. The mixture was then introduced with a spatula into the aluminum cavity in order to test the adhesion of the gel thus produced.

The results below show the variation of the adhesion and the adhesion energy of the gel in the event of the introduction of sucroester S11 up to a percentage of 50% into the hydroxyapatite gel with a nylon mobile device.

| | | | Adhesion | Adhesion | | |
|---|---|---|---|---|---|---|
| | Peak | Area | strength in | energy in | | |
| Gel S11 | in grams | in g/s | Newtons | kJ/m² | N/mm² | N/cm² |
| 0% control | 292.73 | 71.65 | 2.8716813 | 0.000447698 | 0.00914548 | 0.91454818 |
| 3% | 228.5 | 71.08 | 2.241585 | 0.000444137 | 0.00713881 | 0.71388057 |
| 10% | 272.32 | 100.81 | 2.6714592 | 0.000629902 | 0.00850783 | 0.85078318 |
| 20% | 650.39 | 186.33 | 6.3803259 | 0.001164266 | 0.02031951 | 2.03195092 |
| 30% | 647.25 | 291.23 | 6.3495225 | 0.001819724 | 0.02022141 | 2.02214092 |
| 40% | 427.85 | 221.38 | 4.1972085 | 0.001383272 | 0.01336691 | 1.33669061 |
| 50% | 333.1 | 155.92 | 3.267711 | 0.000974252 | 0.01040672 | 1.04067229 |
| Adhesive | 461.45 | 2968.35 | 4.5268245 | 0.018547461 | 0.01441664 | 1.44166385 |

### Formulation 2

A mixture of 2 grams of hydroxyapatite powder, 2 grams of water and the suitable amount of sucroester S11 (corresponding to the defined percentage) was prepared. The mixture was then introduced in the syringe to be injected into water in order to measure the diameter.

The results below show the variation of the swelling of the paste in the event of the introduction of sucroester S11 up to a percentage of 50% for non-calcined hydroxyapatite <200 μm.

Days of variation

| | 0 | | 1 | | 2 | | 3 | | 6 | | 10 | |
|---|---|---|---|---|---|---|---|---|---|---|---|---|
| % S11 | Ø | S | Ø | S | Ø | S | Ø | S | Ø | S | Ø | S |
| 10 | 1.91 | 2.87 | 2.69 | 5.68 | 2.88 | 6.51 | 2.88 | 6.51 | 2.48 | 4.83 | 2.45 | 4.71 |
| 20 | 2 | 3.14 | 3.04 | 7.26 | 3.99 | 12.50 | 3.68 | 10.64 | 3.22 | 8.14 | 3.55 | 9.90 |
| 30 | 1.98 | 3.08 | 3.6 | 10.18 | 4.62 | 16.76 | 5.02 | 19.79 | 3.96 | 12.32 | 3.8 | 11.34 |
| 40 | 1.95 | 2.99 | 4.34 | 14.79 | 5.53 | 24.02 | 5.45 | 23.33 | 4.7 | 17.35 | 4.21 | 13.92 |
| 50 | 2 | 3.14 | 5.6 | 24.63 | 6.08 | 29.03 | 7.25 | 41.28 | 6.4 | 32.17 | 4.51 | 15.98 |

Ø = diameter of section (in mm)
S = surface area of section (en mm²)

A mixture of 2 grams of hydroxyapatite powder, 2 grams of water and the suitable amount of sucroester S11 (corresponding to the defined percentage) was prepared. The mixture was then introduced with a spatula into the aluminum cavity in order to test the adhesion of the paste thus produced. The results below show the variation of the adhesion and adhesion energy of the paste in the event of the introduction of sucroester S11 up to a percentage of 40% for non-calcined hydroxyapatite <200 μm with a nylon mobile device.

| | | | Adhesion | Adhesion | | |
|---|---|---|---|---|---|---|
| | Peak | Area | strength in | energy in | | |
| | in g | in g/s | Newtons | kJ/m² | N/mm² | N/cm² |
| Control | 79.988 | 45.58 | 0.78468228 | 0.000284802 | 0.00249899 | 0.24989882 |
| 10% | 152.67 | 85.18 | 1.4976927 | 0.000532239 | 0.00476972 | 0.4769722 |
| 20% | 360.4 | 601.809 | 3.535524 | 0.003760348 | 0.01125963 | 1.12596306 |
| 30% | 449.77 | 364.71 | 4.4122437 | 0.002278857 | 0.01405173 | 1.40517315 |
| 40% | 327.12 | 149.36 | 3.2090472 | 0.000933262 | 0.0102199 | 1.02198955 |
| Adhesive | 461.45 | 2968.35 | 4.5268245 | 0.018547461 | 0.01441664 | 1.44166385 |

### Formulation 3:

A mixture of 3 grams of hydroxyapatite gel and the suitable amount of sucrose palmitate displaying an HLB equal to 16, hereafter referred to as sucroester P16 (corresponding to the defined percentage) was prepared. The mixture is then introduced with a spatula into the aluminum cavity in order to test the adhesion of the gel thus produced.

The results below show the variation of the adhesion and the adhesion energy of the gel in the event of the introduction of sucroester P16 up to a percentage of 40% into the hydroxyapatite gel with a nylon mobile device.

| | | | Adhesion | Adhesion | | |
|---|---|---|---|---|---|---|
| | | Area | strength in | energy in | | |
| | Peak in g | in g/s | Newtons | kJ/m² | N/mm² | N/cm² |
| Gel | 292.73 | 71.65 | 2.8716813 | 0.000447698 | 0.00914548 | 0.91454818 |
| 20% | 628.95 | 4769.94 | 6.1699995 | 0.02980453 | 0.01964968 | 1.96496799 |
| 30% | 711.8 | 4250.89 | 6.982758 | 0.026561294 | 0.02223808 | 2.2223808 |
| 40% | 877.3 | 6873.31 | 8.606313 | 0.042947243 | 0.02740864 | 2.74086401 |
| Adhesive | 461.45 | 2968.35 | 4.5268245 | 0.018547461 | 0.01441664 | 1.44166385 |

### Formulation 4:

A mixture of 2 grams of hydroxyapatite powder, 2 grams of water and the suitable amount of sucroester P16 (corresponding to the defined percentage) was prepared. The mixture was then introduced with a spatula into the aluminum cavity in order to test the adhesion of the paste thus produced.

The results below show the variation of the adhesion and adhesion energy of the paste in the event of the introduction of sucroester P16 up to a percentage of 40% for non-calcined hydroxyapatite <200 μm with a nylon mobile device.

| | | | Adhesion | Adhesion | | |
|---|---|---|---|---|---|---|
| | Peak | Area | strength in | energy in | | |
| | in g | in g/s | Newtons | kJ/m² | N/mm² | N/cm² |
| Control | 79.988 | 45.58 | 0.78468228 | 0.000284802 | 0.00249899 | 0.24989882 |
| 20% | 228.26 | 80.24 | 2.2392306 | 0.000501372 | 0.00713131 | 0.71313076 |
| 30% | 581.52 | 2083.4 | 5.7047112 | 0.013017932 | 0.01816787 | 1.81678701 |
| 40% | 228.32 | 83.1 | 2.2398192 | 0.000519243 | 0.00713318 | 0.71331822 |
| Adhesive | 461.45 | 2968.35 | 4.5268245 | 0.018547461 | 0.01441664 | 1.44166385 |

### Formulation 5:

A mixture of 3 grams of hydroxyapatite gel and the suitable amount of sucroester S11 (corresponding to the defined percentage) was prepared. The mixture was then introduced with a spatula into the aluminum cavity in order to test the adhesion of the gel thus produced.

The results below show the variation of the adhesion and the adhesion energy of the gel in the event of the introduction of sucroester S11 up to a percentage of 50% into the hydroxyapatite gel with a bone mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 238.1 | 148.18 | 2.335761 | 0.00092589 | 0.00743873 | 0.743873 |
| 10% | 308 | 65.76 | 3.02148 | 0.0004109 | 0.00962255 | 0.962255 |
| 20% | 296.1 | 75.89 | 2.904741 | 0.00047419 | 0.00925077 | 0.92577 |
| 30% | 349.4 | 411.6 | 3.427614 | 0.00257184 | 0.01091597 | 1.091597 |
| 40% | 186.1 | 42.2 | 1.825641 | 0.00026368 | 0.00581414 | 0.581414 |
| 50% | 263.6 | 87.95 | 2.585916 | 0.00054955 | 0.0082354 | 0.82354 |
| Adhesive | 426.4 | 491.93 | 4.182984 | 0.00307378 | 0.01332161 | 1.332161 |

### Formulation 6:

A mixture of 3 grams of hydroxyapatite gel and the suitable amount of sucroester P16 (corresponding to the defined percentage) was prepared. The mixture was then introduced with a spatula into the aluminum cavity in order to test the adhesion of the gel thus produced.

The results below show the variation of the adhesion and adhesion energy of the gel in the event of the introduction of sucroester P16 up to a percentage of 50% into the hydroxyapatite gel with a bone mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 238.1 | 148.18 | 2.335761 | 0.00092589 | 0.00743873 | 0.743873 |
| 10% | 253.6 | 117.1 | 2.487816 | 0.00073169 | 0.00792298 | 0.792298 |
| 20% | 652.4 | 6101.72 | 6.400044 | 0.03812603 | 0.02038231 | 2.038231 |
| 30% | 668.4 | 4377.69 | 6.557004 | 0.02735359 | 0.02088218 | 2.088218 |
| 40% | 406.8 | 136.44 | 3.990708 | 0.00085253 | 0.01270926 | 1.270926 |
| 50% | 428.9 | 344.34 | 4.207509 | 0.00215158 | 0.01339971 | 1.339971 |
| Adhesive | 426.4 | 491.93 | 4.182984 | 0.00307378 | 0.01332161 | 1.332161 |

### Formulation 7:

A mixture of 2 grams of hydroxyapatite powder, 2 grams of water and the suitable amount of sucroester S11 (corresponding to the defined percentage) was prepared. The mixture was then introduced with a spatula into the aluminum cavity in order to test the adhesion of the paste thus produced.

The results below show the variation of the adhesion and the adhesion energy of the paste in the event of the introduction of sucroester S11 up to a percentage of 40% for non-calcined hydroxyapatite <200 μm with a bone mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 45.6 | 93.95 | 0.447336 | 0.00058704 | 0.00142464 | 0.142464 |
| 10% | 102.1 | 39.85 | 1.001601 | 0.000249 | 0.00318981 | 0.318981 |
| 20% | 167.8 | 40.46 | 1.646118 | 0.00025281 | 0.00524241 | 0.524241 |
| 30% | 184.8 | 34.56 | 1.812888 | 0.00021594 | 0.00577353 | 0.577353 |
| 40% | 441.5 | 106.19575 | 4.331115 | 0.00066355 | 0.01379336 | 1.379336 |
| Adhesive | 426.4 | 491.93 | 4.182984 | 0.00307378 | 0.01332161 | 1.332161 |

### Formulation 8:

A mixture of 2 grams of hydroxyapatite, 2 grams of water and the suitable amount of sucroester P16 (corresponding to the defined percentage) was prepared. The mixture was then introduced with a spatula into the aluminum cavity in order to test the adhesion of the paste thus produced.

The results below show the variation of the adhesion and the adhesion energy of the paste in the event of the introduction of sucroester P16 up to a percentage of 40% for non-calcined hydroxyapatite <200 μm with a bone mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 45.6 | 93.95 | 0.447336 | 0.00058704 | 0.00142464 | 0.142464 |
| 10% | 336.6 | 1020.47 | 3.302046 | 0.00637631 | 0.01051607 | 1.051607 |
| 20% | 429.1 | 861.6 | 4.209471 | 0.00538363 | 0.01340596 | 1.340596 |
| 30% | 529.9 | 847.23 | 5.198319 | 0.00529384 | 0.01655516 | 1.655516 |
| 40% | 635.5 | 1021.71 | 6.234255 | 0.00638406 | 0.01985432 | 1.985432 |
| Adhesive | 426.4 | 491.93 | 4.182984 | 0.00307378 | 0.01332161 | 1.332161 |

### Formulation 9:

A mixture of 3 grams of hydroxyapatite gel and the suitable amount of sucroester S11 (corresponding to the defined percentage) was prepared. The mixture was then introduced with a spatula into the aluminum cavity in order to test the adhesion of the gel thus produced.

The results below show the variation of the adhesion and the adhesion energy of the gel in the event of the introduction of sucroester S11 up to a percentage of 30% in the hydroxyapatite gel with a stainless steel mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 215.4 | 94.74 | 2.113074 | 0.00059 | 0.00673 | 0.673 |
| 20% | 292.5 | 66.98 | 2.869425 | 0.00042 | 0.00914 | 0.914 |
| 30% | 390.22 | 108.5 | 3.8280582 | 0.00068 | 0.01219 | 1.219 |
| Adhesive | 908.8 | 2855.91 | 8.915328 | 0.01784 | 0.02839 | 2.839 |

### Formulation 10:

A mixture of 3 grams of hydroxyapatite gel and the suitable amount of sucroester P16 (corresponding to the defined percentage) was prepared. The mixture is then introduced with a spatula into the aluminum cavity in order to test the adhesion of the gel thus produced.

The results below show the variation of the adhesion and adhesion energy of the gel in the event of the introduction of sucroester P16 up to a percentage of 30% in the hydroxyapatite gel with a stainless steel mobile device.

| | | | Adhesion | Adhesion | | |
|---|---|---|---|---|---|---|
| | | | strength in | energy in | | |
| | Peak in g | Area in g/s | Newtons | kJ/m² | N/mm² | N/cm² |
| Control | 215.4 | 94.74 | 2.113074 | 0.00059 | 0.00673 | 0.673 |
| 20% | 1362.5 | 5562.9 | 13.366125 | 0.03476 | 0.04257 | 4.257 |
| 30% | 922.3 | 5987.77 | 9.047763 | 0.03741 | 0.02881 | 2.881 |
| Adhesive | 908.8 | 2855.91 | 8.915328 | 0.01784 | 0.02839 | 2.839 |

1. Calcium phosphate-based formulation for bone filling, characterised in that it comprises at least one adjuvant giving adhesion and swelling properties.

2. Formulation according to claim 1 wherein said at least one adjuvant is selected from the sugar and sugar derivative group.

3. Formulation according to claim 2 wherein the sugars and sugar derivatives are preferentially monosaccharides, disaccharides, oligosaccharides and polysaccharides and derivatives thereof.

4. Formulation according to claim 3 wherein the sugar derivatives are preferentially sugar-derived surfactants selected from the group of sorbitan esters, sucrose fatty esters, sucroglycerides, alkylpolyglucosides and alkylpolyglycosides.

5. Formulation according to claim 4 wherein the sugar-derived surfactants are preferentially sucrose fatty esters.

6. Formulation according to claim 5, comprising 0.01 to 55% of sucrose fatty esters by weight in the final formulation.

7. Formulation according to claim 5, comprising 10 to 40% of sucrose fatty esters by weight in the final formulation.

8. Formulation according to claim 1, wherein the calcium phosphates have a Ca/P atomic ratio between 0.4 and 2.

9. Formulation according to claim 8, wherein the calcium phosphates have a Ca/P atomic ratio between 1.4 and 1.8.

10. Formulation according to claim 9, wherein the calcium phosphates are used in powder form.

11. Formulation according to claim 10, wherein the calcium phosphate powder has a particle size distribution between 1 and 500 μm.

12. Formulation according to claim 1, wherein the calcium phosphates are used in gel form.

13. Formulation according to claim 1, comprising 15 to 99.99% of calcium phosphate.

14. Formulation according to claim 1, wherein the formulation adhesion energy is increased up to 60 times greater than a calcium phosphate-based formulation free from adjuvant.

15. Formulation according to claim 1, wherein the formulation swelling power is increased up to 4 times greater than a calcium phosphate-based formulation free from adjuvant.
